(19) United States
(12) Patent Application Publication
(10) Pub. No.: US 2004/0098003 A1
(43) Pub. Date: May 20, 2004

(54) Pliers-like tool and process for curing phimosis

(76) Inventor: Abe Nishiki, Yamagata-ken (JP)

Correspondence Address: Clyde I. Coughenour, 16607 Sutton Place, Woodbridge, VA, 22191, US

(21) Appl. No.: 10/687,748

(22) Filed: Oct. 20, 2003

(30) Foreign Application Priority Data
Oct. 21, 2002 (JP) 2002-306198

(51) Int. Cl. A61B 17/32

(52) U.S. Cl. 606/167; 606/118

(57) ABSTRACT

A pliers-like tool has handles and upturned blades. Moving the handles together spreads the upturned blades. With the blades inserted into a foreskin opening, prior to spreading the blades away from each other, the foreskin can be stretched. A ratchet extending between the handles retains the blades in a spread apart condition within the foreskin to expand it. Stretching the foreskin opening for about 30 minutes twice a day for 2-3 weeks is enough to expand it to cure phimosis.

### BACKGROUND OF THE INVENTION

1. Field of the Invention

A pliers-like tool spreads the foreskin to stretch it and cure phimosis.

2. Description of Related Art

Although true phimosis, where the whole penis is covered with foreskin and the glans are not exposed at all, even at the time of erection, can be cured through an orthopedic operation that cuts the excess foreskin and widens the foreskin opening, there has been the problem that, in addition to the fact that the operation cost is significantly high, the cost of aftercare and medicine mount up, making the monetary burden expensive.

A phimosis operation runs the risk that cutting too much foreskin may cause tension in the foreskin at the time of erection, that the sutured section of the foreskin may become ugly in a keloidal state, etc., and that ejaculation may become impossible by cutting off a part of the erogenous zone together with the foreskin or damaging the sexual feeling transmitting nerve. The pain will continue about two weeks after an operation, and because the bandage cannot be removed, bathing and urinating become difficult, handicapping the life of the phimosis patient. Many phimosis patients are too ashamed to have an operation, anguishing and suffering with it secretly.

According to a phimosis manual “Phimosis Counseling Room for Children” true phimosis is caused by the fact that the foreskin ring at the foreskin tip enveloping the glans is too small, and there are descriptions that “if the foreskin ring is expanded using forceps, the glans can be exposed from the opening.” and that “it is desired that a phimosis curer which can be easily used by non-specialists be commercially sold.”

When the present inventor manufactured a phimosis curer with which a phimosis patient can cure phimosis by himself and tried it with a patient, in about 2˜3 weeks of using it twice a day, in the morning and afternoon for 30 minutes each, the foreskin ring expanded, its opening widened, and the glans became completely exposed.

The handle grips have to be held for as long as 30 minutes each use, and they need to be used twice a day continuously for 2˜3 weeks. Hand holding the grips becomes tiresome. Because of this the user becomes reluctant to continue treatment and there have been cases where the user stops before any noticeable cure appears.

There has been a problem that in spite of the fact that a curing effect is not achieved unless the grips are squeezed firmly enough to feel some pain from the foreskin ring, there is a tendency to relieve pain when self treating. The patient tends to loosen the force created by the hand so that there will be no pain felt, and as a result not enough curing is achieved.

If the hand slips while squeezing the grips, the blades suddenly close under the elastic force of the spring applied to the blades, possibly hurting the penis glans and foreskin interior. The glans epidermis is the thinnest of all the body surfaces and becomes easily damaged, and it is one of the parts where nerves are most concentrated. As a result, severe pain can be inflicted.

When the opening is widened by squeezing the tool to expand the foreskin ring, if the foreskin slides off the blades due to its elasticity, the foreskin and blades rub against each other and the foreskin may even be torn.

The use of pliers and pliers-like tools is old and there are numerous varieties of them. While most pliers are used in a pinching action, they have been widely used to separate or press outwardly. Examples are to stretch shoes (F. L. Walker & C. F Smith, U.S. Pat. No. 797,541, issued 15 Aug. 1905), to remove or expand piston rings (O. Winter, U.S. Pat. No. 855,031, issued 28 May 1907, and E. H. Kind, U.S. Pat. No. 1,319,007, issued 14 Oct. 1919), spread bottle nipples (G. E. Lovekin, U.S. Pat. No. 1,978,210, issued 23 Oct. 1934), as a dilator (A. G. Schenk, U.S. Pat. No. 4,257,406, issued 24 Mar. 1981), spacing bones (K. L. Cox, U.S. Pat. No. 4,754,746, issued 5 Jul. 1988), cavity creation (Gellman et al, U.S. Pat. No. 6,099,547, issued 8 Aug. 2000), removing light bulbs (B. L. Christie, U.S. Pat. No. 5,371,658, issued 6 Dec. 1994), etc.

### SUMMARY OF THE INVENTION

The phimosis curer is a pliers-like tool constructed so that when handle grips are squeezed together against the elastic force of a spring, two jaws open and a pair of blades installed essentially perpendicularly to the tips of the jaws move away from each other. The pair of blades are inserted into the foreskin opening and the grips are squeezed to spread the blades and expand the foreskin ring and stretch the foreskin opening.

The objective of the present invention is to make a tool that can be used continuously for a long time without tiring the hand, to effectively expand the foreskin ring to feel some pain for widening the foreskin opening, and eliminate the danger of hurting the glans or foreskin.

The present invention is characterized by the fact that in a phimosis curer a pair of blades move apart when hand grips consisting of two shafts or handles are squeezed together. The blades are inserted into the foreskin opening of a phimosis and the foreskin opening is widened. The curer is equipped with a ratchet mechanism between the handles that allows the grips to move freely together, according to the desired force applied to the grips, and to fix the grips inseparable against any force applied in the direction of separating the grips.

A pair of blades are inserted into the foreskin opening of a phimosis and the grips are squeezed together, causing the blades to separate from each other. When the handle grips are squeezed together the grips move freely as close as desired over the ratchet mechanism, the blades move apart from each other, the foreskin ring is expanded by these blades, and the foreskin opening is widened. When the foreskin opening is increased by the blades, a force to move the blades closer to each other is created by the elasticity of the foreskin ring, and with any other force tending to move the grips apart from each other prevented. The grips become fixed inseparably by the ratchet mechanism. Even if the hand force squeezing the grips is released, the blades never close, and the foreskin ring stays expanded even without continued squeezing on the grips, and the foreskin opening is maintained.

If a large-diameter section with a curved circumferential face is formed on the blade tips, the foreskin will not slip off of the blades and rub, and the force will not be concentrated in one area or part inside the foreskin. It will not wear out or develop a weal even after long-term use.

### BRIEF DESCRIPTION OF THE DRAWINGS

FIG. 1 is an exploded perspective view of the phimosis curer of the present invention.

FIG. 2(a) is a front view and (b) a top view showing the shape of the blades.

FIG. 3(a), (b), and (c) are top views showing the different open/close states.

FIG. 4 is a rear view of the phimosis curer.

FIG. 5 is a top view of a first modified phimosis curer

FIG. 6 is a prospective view of a second modified phimosis curer.

### DESCRIPTION OF THE PREFERRED EMBODIMENTS

A phimosis curer 1 of the present embodiment is formed in a pliers shape where a pair of blades 3R and 3L, having first inner ends and second outer ends, move apart from each other when handle grips 2R and 2L, having first outer ends and second inner ends, are squeezed together after being inserted into the foreskin opening of a phimosis to expand the foreskin and widen the foreskin opening ring.

The handle grips 2R and 2L are formed as a unit with jaws 4R and 4L, respectively, having first inner ends and second outer ends. The jaws are rotatably joined via a fulcrum pin 5 formed with blades 3R and 3L erected upwardly perpendicularly on the respective outer ends. Embedded with the fulcrum 5 is a spring (not shown) that applies a force in the direction to open the grips 2R and 2L, and close the blades 3R and 3L.

FIG. 2(a) is a front view of the phimosis curer 1 seen from the front or tip side, and FIG. 2(b) is a front top view showing the tip of the jaw. Formed on the upper tips of the blades 3R and 3L are enlarged ball hooks, large-diameter sections, 3a where the foreskin ring is secured so that the foreskin will not fall or slip off the blades 3R and 3L when the foreskin opening of a phimosis is widened. The ball hooks 3a are formed in a near-spherical shape that forms spaces 3b˜3d in the tip side and both the front and rear sides when the blades 3R and 3L are closed, so that the penis glands and foreskin interior are not sandwiched or pinched. The faces of the blades 3R and 3L and the ball hooks 3a are finished, smooth curved surfaces treated with a plating process of a metal, or coated with a resin film inert and not harmful to the human body, so that it will not damage the foreskin when it is inserted into the foreskin opening to widen it.

Installed on the handle grips 2R and 2L is a ratchet mechanism 6 that permits the grips 2R and 2L to freely move together as close to each other as desired and that can hold the handle grips 2R and 2L inseparable against a force operating to separate them from each other. When the blades 3R and 3L are inserted into the foreskin opening and the grips 2R and 2L are squeezed together, because the handle grips 2R and 2L move toward each other, they freely pass together unaffected by the ratchet mechanism 6, and the blades 3R and 3L open by moving away from each other. Thus, the foreskin ring is expanded and the foreskin opening widened. At this time, the opening between the blades 3R and 3L is selected to be about 4˜5 cm, the distance necessary for exposing the penis glans.

When a force moves the blades 3R and 3L together, such as by the elasticity of the expanded foreskin ring or other force to move the handle grips 2R and 2L apart, the handle grips 2R and 2L become secured together by engagement of the ratchet mechanism 6, and therefore the blades 3R and 3L do not move toward each other.

Ratchet mechanism 6 is equipped with a pawl 7, pivotably held by a second pivot pin on one handle grip 2R, and a rack 8, attached to the other handle grip. The rack 8 is designed to engage with the pawl 7 when the grips 2R and 2L move away from each other.

The curved rack 8 having an upper surface, a lower surface, first end, second end, first and second side, is pivotably supported at its first end on the handle grip 2L by a first pivot pin 9, that receives a force or bias from a spring 10. The rack has cogs 11 formed on its first side outer edge that mesh with the pawl 7 when the grips 2R and 2L rotate in the direction to separate or open them (the direction in which the blades 3R and 3L close). The spring 10 urges the cogs on the rack toward the pawl 7.

A projection 12 is installed on one of the handle grips. The projection pops out and comes in contact with the bottom or lower surface of the curved rack 8 by a plate spring 13 with a force applied in the outward projection direction. A stopper 16 is also installed that restricts the fluctuation or pivot angle of the rack so that the projection 12 is always positioned below the curved rack 8, even when the curved rack 8 fluctuates or pivots. The pivot of the curved rack 8 in the arrow B direction is restricted by the pawl 7, and the pivot in the reverse arrow B direction is restricted by the stopper 16.

Formed on the bottom or lower surface of the curved rack 8 in a sliding surface 15 is a guide groove 14 that engages with the projection 12 in the projected state. Engaged together they maintain the rack 8 and the pawl 7 in the disengaged state. The sliding surface 15 maintains the projection 12 in the depressed or “sunk-in” state and, when the projection 12 is not in the guide groove 14 under spring pressure, permits the rack 8 and the pawl 7 to be in the engaged state.

When the projection 12 is engaged in the guide groove 14, because the rack 8 is coupled 2 the projection 12, the rack 8 and the pawl 7 are maintained in the disengaged state against a force applied by the spring 10. If the projection 12 sinks inside the grip 2R, engagement between the guide groove 14 and the projection 12 is released, and the rack 8 contacts with the pawl 7 and is kept in an engaged state by a force applied by the spring 10. The projection 12 is formed in a position where it can be engaged with the guide groove 14 by pivoting the curved rack 8 irrespective of the distance between the blades 3R and 3L.

Formed on the second outer end of the rack 8 is a tapered surface 14a where the projection 12, engaged within the guide groove 14, is pressed into the handle grips when the grips 2R and 2L pivot away from each other. When the grips 2R and 2L open (when the blades 3R and 3L close), engagement between the guide groove 14 and the projection 12 is released.

When squeezing the grips 2R and 2L from the open state, because the projection 12 is held inside the grip 2R by the sliding surface 15 of the rack 8, the blades 3R and 3L can be opened to an arbitrary or selected distance until the pawl 7 and the curved rack 8 of the ratchet mechanism 6 engage.

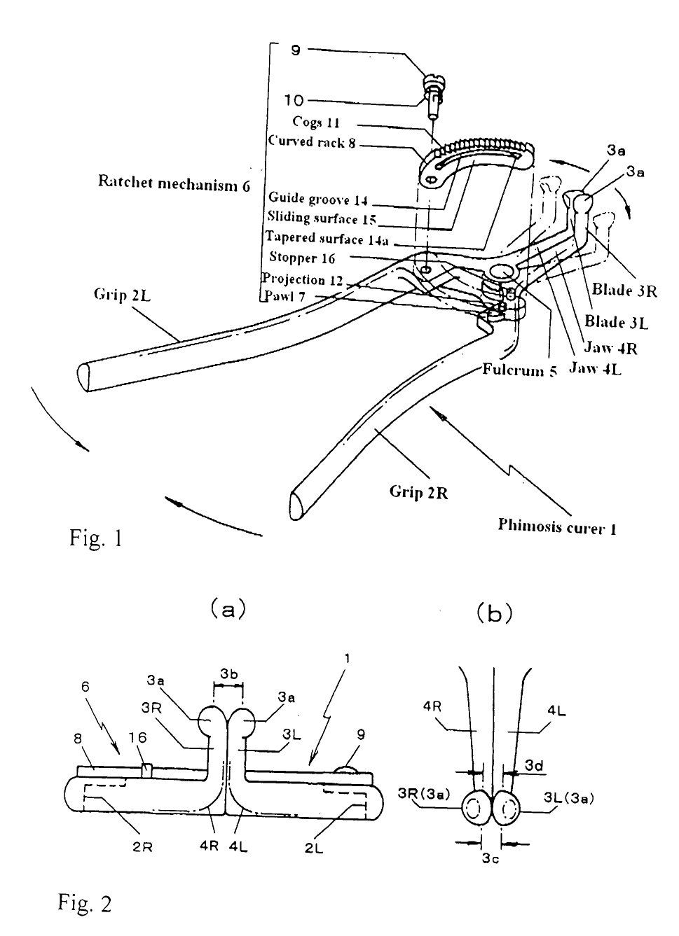

When closing, if the curved rack 8 pivots or fluctuates against the elastic force of the spring 10, the projection 12 eventually engages with the guide groove 14. The curved rack 8 and the pawl 7 are maintained in the disengaged state. The grips 2R and 2L open by the elastic or resilient bias force of a spring (not shown but similar to the spring 10 shown in FIG. 1) embedded in the fulcrum 5, and the blades 3R and 3L close.

In operation, directing the right hand palm upward, the phimosis curer 1 is put on it, the grip 2R is set in contact with the right hand thumb cushion, the little finger and the third finger are put on the grip 2L, and the thumb tip is kept free when holding the tool so that the curved rack 8 can be easily operated.

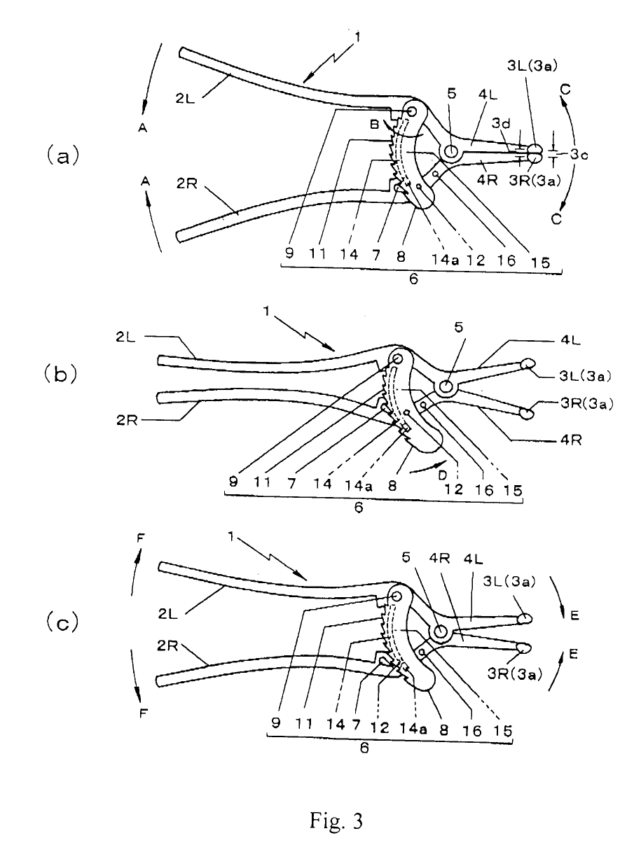

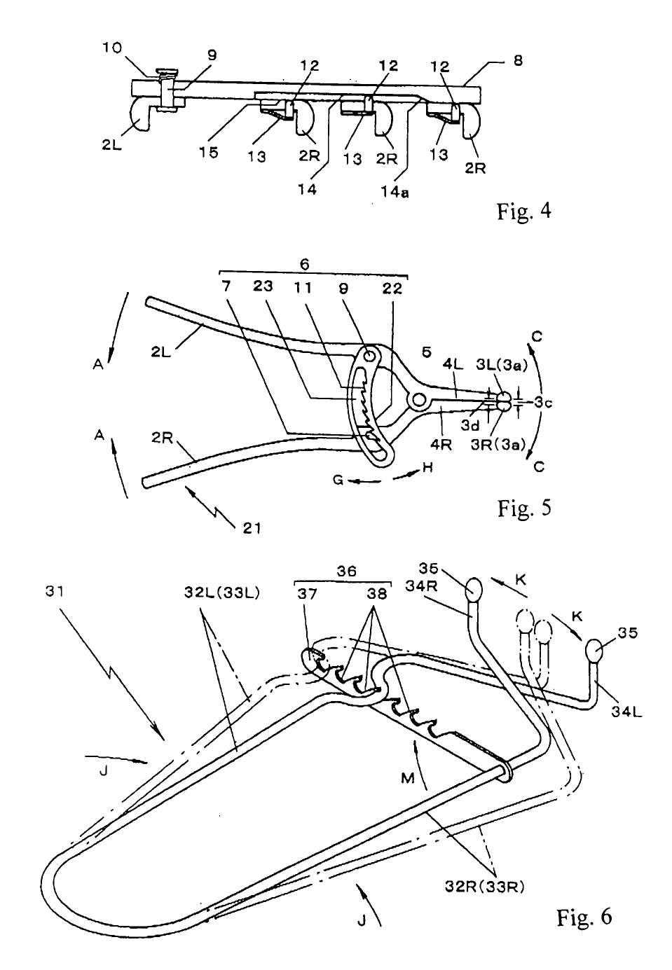

First, as shown in FIG. 3(a), closed blades 3R and 3L are inserted into the foreskin opening of a phimosis, and the grips 2R and 2L are squeezed in the direction of the arrow A until the foreskin ring is hooked over the ball hooks 3a on the outer tips of the blades.

At this time, the grips 2R and 2L can be moved close together. The ratchet mechanism 6, while keeping the curved rack 8 engaged with the pawl 7 under spring applying force in the arrow B direction, permits the pawl to glide over the cogs. Because the blades 3R and 3L move away from each other in the direction of parting, as shown by the arrow C, the foreskin ring becomes expanded, and the foreskin opening is further widened.

If the force of the hand is released or loosened when the blades are opened to a certain degree, the elastic force of the foreskin ring tends to close the blades 3R and 3L and, along with this, a spring force operates in the direction of making the grips 2R and 2L part from each other.

At this time, because the curved rack 8 and the pawl 7 are engaged, as shown in FIG. 3(b), the grips 2R and 2L are fixed against rotation and therefore the blades 3R and 3L are also fixed so that the foreskin ring is expanded and the foreskin opening is kept widened without the need to continue holding the grips 2R and 2L with the hand. Because the blades 3R and 3L can be fixed at an optimal distance by the ratchet mechanism 6, the curing process works well. Because the foreskin ring is hooked to the ball hook 3a, when it is expanded, the foreskin will not slip off of the blades 3R and 3L. Also, because the ball hook 3a is formed in a ball or near-spherical shape, the force does not concentrate on one area or part inside the foreskin, and therefore it will never wear out or develop a weal even after long-term use.

After leaving it for 30 minutes, if the tip of the curved rack 8 is pushed with the thumb in the arrow D direction in FIG. 3(b), against the elastic force of the spring 10, and let fluctuate or pivot so that the guide groove 14 of the curved rack 8 is positioned right above the projection 12, as shown in FIG. 3(c), engagement between the curved rack 8 and the pawl 7 is released and the projection 12 pops out to be engaged with the guide groove 14.

With the curved rack 8 and the pawl 7 maintained in the disengaged state, the grips 2R and 2L open by rotating away from each other, as shown by the arrow F, by a spring, such as 10, embedded in the fulcrum 5, and the blades 3R and 3L close by rotating closer to each other in the arrow E direction as shown in FIG. 3(c).

Note that at the moment engagement between the curved rack 8 and the pawl is released, even if the blades 3R and 3L suddenly close, because the ball hooks 3a that hook the foreskin ring are formed in a near-spherical shape, with spaces 3b˜3d formed in its tip in both the front and rear sides, neither the penis glans nor foreskin inside face will be sandwiched or pinched by the ball hooks 3a.

Once the blades 3R and 3L are about to completely close, the projection 12 is guided by a decreasing depth tapered surface 14a, formed on the outer end of the guide groove 14, down until it is sunk or depressed inside the handle grip 2R.

With release of engagement between the projection 12 and the guide groove 14, the curved rack 8 engages with the pawl 7 by the elastic force of the spring 10, and the projection 12 is kept sunk in the handle grip 2R by the sliding surface 15 of the rack 8.

Because the blades 3R and 3L are completely closed and the pawl 7 and the curved rack 8 of the ratchet mechanism are engaged, there is no need to switch the direction of the ratchet mechanism 6 the next time a curing treatment is performed.

When the phimosis curer 1 of the invention was tried by phimosis patients, using it twice, in the morning and afternoon, every day for about 30 minutes each, without tiring one hand, the foreskin opening was widened by expanding the foreskin ring effectively to feel some pain. The penis glans became completely exposed in 2˜3 weeks.

In this way, the penis glans can be exposed in a way close to the natural growth process where the penis glans widen the foreskin ring along with the growth of the penis without performing any surgical operation and the patient is not forced to undergo the operation risks and a handicapped life.

Because the open/close operation of the blades 3R and 3L can be performed extremely smoothly and safely by the spring embedded in the fulcrum 5 and the ratchet mechanism 6 in this example, there is little danger of hurting the penis glans that could occur with an excess force operating the grips 2R and 2L. Because the ratchet mechanism 6 is installed in the phimosis curer 1, the blades 3R and 3L never close suddenly while in use, and therefore it will not hurt the penis glans that are one of the thinnest and most fragile parts having the most nerves concentrated on the body surface. Because the ball hooks 3 are formed on the outer tips of the blades 3R and 3L, the foreskin will never be hurt by being rubbed, because spaces 3b18 3d are formed with the ball hooks 3a, the penis glans will not be hurt by being sandwiched or pinched by the ball hooks 3a.

Shown in FIG. 5 is a second embodiment of the phimosis curer of the present invention, where the same designations are assigned to the parts in common with those in FIGS. 1˜4. In a phimosis curer 21 of this embodiment, a slit or slot 23 is formed along the central interior length of a curved rack 22 of a ratchet mechanism 6, and cogs 11 which engage with a pawl 7 are formed inside the slit 23.

The slit 23 is formed with a width such that the curved rack 22 can pivot or fluctuate so that the cogs 11 can engage/disengage with the pawl 7. If the curved rack 22 is pushed in the arrow G direction, the cogs 11 and the pawl 7 engage with each other so that grips 2R and 2L do not rotate in the opening direction (the direction in which the blades close). If the curved rack 22 is pushed in the arrow H direction, engagement between the cogs 11 and the pawl 7 is released so that the grips 2R and 2L and blades 3R and 3L can rotate in either direction.

When holding the grips 2R and 2L with the right hand, the cogs 11 and the pawl 7 can be engaged/disengaged by operating the end of the curved rack 22 in the arrows G and H directions using the thumb.

Although it is desirable to install a spring in the pin 9 that applies a force in the direction to engage the curved rack 22 with the pawl 7, the pin 9 supports the curved rack 22 freely so as to be pivotable, this spring does not necessarily need to be installed.

The phimosis curer 1 is held in the right hand with the palm directed upward, the grip 2R is put on the right hand thumb cushion, the little finger and the third finger are put on the grip 2L, and the thumb tip is kept free so that the curved rack 22 can be freely operated.

Then, the grips 2R and 2L are squeezed as shown with the arrow A as the closed blades 3R and 3L are inserted into the foreskin opening of a phimosis, and the foreskin ring is hooked onto the ball hooks 3a at the tip. At this time, because the grips 2R and 2L can be moved in the direction to bring them closer and move the blades 3R and 3L away from each other, as shown with the arrow C, the foreskin ring is expanded to widen the foreskin opening.

When it is widened to a desired degree, if the curved rack 22 is fluctuated or rotated by the thumb in the arrow G direction to engage with the pawl 7, the grips 2R and 2L become fixed from rotatation, and therefore the blades 3R and 3L also become fixed, and the foreskin ring is expanded and the foreskin opening is maintained without continuing to hold the grips 2R and 2L with a hand. Because the blades 3R and 3L can be fixed at the optimal distance apart for an individual, by the ratchet mechanism 6, the curing effect is good.

After leaving the phimosis curer for 30 minutes, if the tip of the curved rack 22 is pushed with the thumb in the arrow H direction, because the curved rack 22 rotates or fluctuates and its engagement with the pawl 7 is released, the grips 2R and 2L rotate in the opposite direction of the arrow A apart from each other, and the blades 3R and 3L rotate in the opposite direction of the arrow C to become closer together.

In this case, too, if ball hooks 3a are formed on the tips or outer ends of the blades 3R and 3L, even if the blades 3R and 3L suddenly close at the moment when engagement between the curved rack 22 and the pawl 7 is released, neither the penis glans, epidermis nor the foreskin inside surface will be sandwiched or hurt by the ball hooks 3a.

Shown in FIG. 6 is a third embodiment. In a phimosis curer 31 of this embodiment, handle grips 32R and 32L consist of two legs 33R and 33L which are piano wires (spring elements) of 3˜4 mm in diameter permanently bent in a near “U” shape or “V” shape but extending away from each other to retain a spring force tending to separate the legs. Both legs 33R and 33L are permanently bent so that their ends cris-cross each other forming jaws and are permanently bent up into blades having tip ends in a hook shape 35. The tip parts 35 are on the ends of the outwardly extending blades 34R and 34L. The ball hooks 35 (large-diameter sections) hook the foreskin ring so that the foreskin will not drop off the blades 34R and 34L when the foreskin opening of a phimosis is being widened.

The phimosis curer 31 consists of one integral spring element that is bent beyond its elastic limit to form the grips, legs, blades, and can also form the hook balls. The spring element extends between and joins the first and second handle grips and extends beyond the grips to form the legs and blades. The handle grips are biased away from each other by their commonly formed “V” or “U” shaped spring element by limiting the permanent bend.

Installed on the grips 32R and 32L is a ratchet mechanism 36 that permits the grips 32R and 32L to be placed as close to each other as desired according to a force operating in the direction of making these grips closer together and fix the grips inseparable against a force operating in the direction to part the grips from each other.

The ratchet mechanism 36 is equipped with a flat rack 37, having a first side edge and a second side edge, attached rotatably to one first grip 32R, and formed on the rack 37 first side edge are cogs 38 that engage with the other second handle grip 32L when the grips 32R and 32L move in the direction to separate from each other.

The phimosis curer 31 is held in the right hand palm directed upward, the grip 32R is put on the right hand thumb cushion, the little finger and the third finger are put on the grip 32L, and the thumb tip is kept free so that the flat rack 37 can be freely operated. Then, the grips 32R and 32L are squeezed as shown with the arrow J after the closed blades 34R and 34L are inserted into the foreskin opening of a phimosis, and the foreskin ring is hooked to the ball hooks 35 at the tip of the blades. At this time, because the grips 32R and 32L can be moved closer together, the blades 34R and 34L move apart from each other as shown by the arrow K, the foreskin ring is expanded to widen the foreskin opening.

Then, when it is widened to a desired degree, if the flat rack 37 is fluctuated or rotated by the thumb in the arrow M direction to engage with the grip 32L, the grips 32R and 32L become fixed against separation, even if the force of the hand holding the grips 32R and 32L is released or loosened, the blades 34R and 34L will not close, and the foreskin ring is expanded and the foreskin opening is widened as the spacing is maintained. Because the blades 34R and 34L can be fixed at the optimal distance by use of the ratchet mechanism 36, according to individual needs, the curing effect is good.

After leaving it fixed at the optimum spacing for 30 minutes, if the grips 32R and 32L are squeezed a little in the arrow J direction, because the force applied from the grip 32L to the cogs 38 of the rack 37 is loosened, the rack 37 rotates by gravity in the opposite direction of the arrow M to come off, or by taking it off with the index finger, engagement with the grip 32L can be easily released.

Thus, the grips 32R and 32L rotate in the opposite direction of the arrow J to separate from each other, and at the same time the blades 34R and 34L come closer together in the opposite direction of the arrow K.

With the ball hooks 35 formed on the tips of the blades 34R and 34L, even if the blades 3R and 3L suddenly close at the moment the engagement between the rack 37 and the grip 32L is released, neither the penis glans, epidermis nor the foreskin inside surface will be sandwiched or pinched by the ball hooks 35.

The ratchet mechanisms 6 and 36 are not limited to the specific structures of the embodiments.

Because the blades can be maintained in an open state by the ratchet mechanism, even if the force exerted by hand holding the grips is loosened, it has the excellent effect that the hand never becomes tired even after long hours of its continuous use. The foreskin ring can be expanded effectively to an optimum degree, until there is a feeling of some pain, to widen the foreskin opening, with there being no danger of hurting the penis glans or the foreskin.

It is believed that the construction, operation and advantages of this invention will be apparent to those skilled in the art. It is to be understood that the present disclosure is illustrative only and that changes, variations, substitutions, modifications and equivalents will be readily apparent to one skilled in the art and that such may be made without departing from the spirit of the invention as defined by the following claims.

1. A phimosis curer comprising;
a first handle grip having a first end and a second end and a second handle grip having a first end and a second end;
a first jaw having a first end and a second end and a second jaw having a first end and a second end;
a first blade having a first end and a second end and a second blade having a first end and a second end;
said first handle grip second end and said first jaw first end are joined together;
said second handle grip second end and said second jaw first end are joined together;
said first blade first end and said first jaw second end are joined together;
said second blade first end and said second jaw second end are joined together;
said first blade and said second blade extend upwardly from said first jaw and said second jaw respectively so as to be insertable into a foreskin opening;
said first handle grip and said first jaw are joined to said second handle grip and said second jaw such that moving said first handle grip and said second handle grip toward each other moves said first jaw and said second jaw away from each other;
a ratchet means pivots between said first handle grip and said second handle grip adjacent said first handle grip second end and said second handle grip second end;
said ratchet means is designed to selectively prevent said first handle grip and said second handle grip from moving away from each other;
a first enlarged hook means on said first blade second end and a second enlarged hook means on said second blade second end to prevent said first blade and said second blade from slipping out of a foreskin opening when said first blade and second blade are separated within a foreskin opening.

2. A phimosis curer as in claim 1 including:
a fulcrum pin attaching said first handle grip and said second handle grip together so that said first handle grip and said first jaw can be pivoted relative to said second handle grip and said second jaw.

3. A phimosis curer as in claim 2 wherein:
said ratchet means includes a curved rack with an upper surface, a lower surface, a first side surface and a second side surface, a first end, a second end, cogs, and a pawl;
said curved rack first end is attached to said first handle grip second end by a first pivot pin;
said pawl is attached to said second handle grip second end by a second pivot pin.

4. A phimosis curer as in claim 3 wherein:
said curved rack is provided with a slot along its interior length;
said pawl fits within said curved rack slot;
said slot is wide enough to accommodate said pawl in both the engaged position, against said cogs on said curved rack when said first handle grip and said second handle grip are pivoted toward each other, and the disengaged position, away from said cogs on said curved rack, for permitting said first handle grip and said second handle grip to be pivoted away from each other.

5. A phimosis curer as in claim 3 wherein:
said curved rack is provided with one smooth side surface and one irregular cog surface;
said curved rack irregular cog surface pivots toward said pawl to selectively engage said pawl with said irregular cogs, and away from said pawl to remove said curved rack from contact with said pawl.

6. A phimosis curer as in claim 3 wherein:
said curved rack lower surface has a guide groove;
said second handle grip second end has a spring-loaded projection;
a stop is positioned on said second handle grip second end on said first side surface of said curved rack second end and said pawl is positioned on said second side surface of said curved rack to limit pivotal movement of said curved rack about said first pivot pin to keep said spring-loaded projection against said curved rack lower surface at all times.

7. A phimosis curer as in claim 6 wherein:
said spring-loaded projection extends into said guide groove when said guide groove is positioned above it;
said guide groove controls the pivotal movement of said curved rack about said first pivot pin when said projection is in said guide groove to maintain said pawl disengaged from said cogs on said curved rack.

8. A phimosis curer as in claim 7 wherein:
said guide groove terminates in a decreasing depth taper adjacent to said curved rack second end so that moving said first handle grip and said second handle grip away from each other pushes said projection from said guide groove by pressing said projection into said second handle grip;
resilient means bias said curved rack toward said pawl to maintain said pawl in engagement with said cogs when said projection is released from said guide groove.

9. A phimosis curer as in claim 1 wherein:
said enlarged hook means are in the general shape of a ball with finished smooth curved surfaces and with spaces provided to preclude damage to the penis glands and from pinching the foreskin.

10. A phimosis curer as in claim 9 wherein:
said enlarged hook means are coated with an inert material not harmful to the human body.

11. A phimosis curer as in claim 1 wherein:
said first handle grip and said first jaw and said first blade and said first enlarged hook means are one integral part;
said second handle grip and said second jaw and said second blade and said second enlarged hook means are one integral part.

12. A phimosis curer as in claim 3 wherein:
a bias means with said first pivot pin for biasing said cogs on said ratchet means curved rack toward said pawl.

13. A phimosis curer as in claim 1 wherein:
said first handle grip and said first jaw and said first blade and said first enlarged hook means and said second handle grip and said second jaw and said second blade and said second enlarged hook means are all one integral shaped spring element.

14. A phimosis curer as in claim 13 wherein:
said first handle grip first end and said second handle grip first end are united together;
said first jaw and said second jaw crisscross each other.

15. A phimosis curer as in claim 13 wherein:
said ratchet means has a flat rack having a first side edge and a second side edge;
said flat rack is attached to said second handle grip;
cogs are formed along said flat rack first side edge;
said ratchet means is engaged by selectively placing said first handle grip into one of said cogs on said flat rack first side.

16. A phimosis curer as in claim 2 including:
a spring with said fulcrum pin for biasing said first handle grip and said second handle grip away from each other.

17. A process for curing phimosis comprising:
providing a pliers-like hand tool having a first handle grip and a second handle grip with attached first and second jaws respectively and respective first blade and second blade joined to and extending outwardly from said first jaw and said second jaw so that moving said first handle grip and said second handle grip toward each other moves said first blade and said second blade away from each other;
providing a ratchet means between said first handle grip and said second handle grip;
inserting said first blade and said second blade under a foreskin opening;
squeezing said first handle grip and said second handle grip together moving said first blade and second blade apart stretching said foreskin;
engaging said ratchet means preventing said first handle grip and said second handle grip from separating and said first blade and said second blade from moving closer together;
holding said foreskin in the stretched condition;
retaining said ratchet means engaged and said foreskin stretched for essentially thirty (30) minutes to elongate the foreskin opening;
releasing said ratchet and moving said first blade and said second blade together and removing said blades from under the foreskin opening.

18. A process for curing phimosis as in claim 17 additionally:
providing a first hooking means on the outer end of said first blade and a second hooking means on the outer end of said second blade;
preventing the foreskin from slipping off of said first blade and said second blade by inserting said first hooking means on said first blade and said second hooking means on said second blade under the foreskin opening prior to squeezing said first handle grip and said second handle grip together.

19. A phimosis curer as in claim 18 additionally:
repeating said stretching steps once each morning and once each afternoon for two to three weeks.
